(19) United States
(12) Patent Application Publication
(10) Pub. No.: US 2009/0035802 A1
(43) Pub. Date: Feb. 5, 2009

(54) METHOD FOR DETECTION AND ENUMERATION OF CELL SURFACE MARKERS

(75) Inventors: Jun-keun Chang, Seoul (KR); Chan-il Chung, Uiwang (KR); Ho-young Yun, Seoul (KR); Hyun-woo Bang, Seoul (KR)

Correspondence Address: EDWARDS ANGELL PALMER & DODGE LLP, P.O. BOX 55874, BOSTON, MA, 02205, US

(73) Assignee: Digital Bio Technology, Co., Ltd., Seoul (KR)

(21) Appl. No.: 11/831,049

(22) Filed: Jul. 31, 2007

(51) Int. Cl. C12Q 1/02; C12M 1/00

(52) U.S. Cl. 435/29; 435/288.7

(57) ABSTRACT

Disclosed is a method for detecting and counting cell surface markers using a difference in fluorescence intensities. A fluorescent material having a relatively lower fluorescence intensity is conjugated with one marker and another fluorescent material having a relatively higher fluorescence intensity is conjugated with the other marker. The two markers can be sorted and counted individually and simultaneously from the fluorescence intensity difference.

### TECHNICAL FIELD

The present invention relates to a method for detecting cell surface markers. More particularly, it relates to a method for detecting at least two cell surface markers by using a single detector, and a method for counting cells based on the detection.

### BACKGROUND ART

Flow cytometers have been widely used in various fields including molecular biology, pathology, immunology, or the like. Typically, a flow cytometer includes the following five main components: a fluid containing cells to be analyzed, a light source, a detector/analyzer, an amplifier, and a computer for analysis of the signals. Modern flow cytometers include several lasers and a fluorescence detector.

Flow cytometry is a technique for sorting, counting and examining microscopic particles suspended in a fluid. It can analyze several thousand particles in “real time.” More particularly, it can analyze particles or cells in an emulsion state promptly when each particle or cell passes through a predetermined sensing point and, if desired, can selectively sort a specific part of such particles or cells.

It also allows simultaneous multiparametric analysis of the physical and/or chemical characteristics of a single cell flowing through an optical and/or electronic detection apparatus. In general, such analysis is performed by applying laser beams with a single wavelength to a fluid, and then analyzing the results by using a plurality of detectors for analyzing the light transmitted from the fluid; for example, an FSC (forward scatter) disposed in line with the flow of fluid and several SSCs (side scatters) disposed perpendicular thereto.

Flow cytometry can also carry out analysis of cell volumes, morphological analysis of cells, DNA analysis (cell cycle analysis etc.), RNA analysis, chromosome analysis, analysis of various antigens, or the like.

It is known that infection with HIV results in a decrease in the number of CD4+ cells in blood and a slight increase in the number of CD3 and CD8 cells in blood. It is also known that the sum of the numbers of CD4+ and CD8 cells should normally be the same as the number of CD3 cells (acceptable error: 5-10%).

Many attempts have been made to measure the number of CD4+ T cells for the purpose of diagnosis of HIV/AIDS. One example of the attempts was to count all white blood cells (CD45) and CD4+ cells and measure the relative ratio thereof, or monitor the ratio of [CD4 cell number]/[CD8 cell number]. However, such prior art method has disadvantages. That is, it requires at least two antibodies and two detectors with different (emission) wavelengths. Additionally, it requires a complicated sample preparation step, and thus it is hard to develop a portable (e.g., toaster size) device.

To solve the above-described problems associated with using two antibodies and two detectors of different wavelengths, U.S. Pat. No. 5,627,037, for example, provides a method of using a difference in fluorescence intensities at a single wavelength (range). This method makes it possible to count both cells and bright beads via one-step detection at a single wavelength (range) by detecting a difference between the fluorescence intensities from a cell surface marker and the beads.

However, no prior art has proposed a method for analyzing at least two cell markers separately and simultaneously at a single wavelength range by using a single detector. That means, when at least two kinds of cells exist, different types of fluorescent materials with different emission wavelengths have been used in the art (see FIG. 2).

The information disclosed in this Background Art section is only for enhancement of understanding of the background of the invention and should not be taken as an acknowledgement or any form of suggestion that this information forms the prior art that is already known to a person skilled in the art.

### SUMMARY OF THE INVENTION

It is an object of the present invention to provide a flow cytometry method for detecting at least two markers by a single detector at a single wavelength range.

It is another object of the present invention to provide a method for diagnosis of diseases based on the detection.

It is still another object of the present invention to provide a flow cytometry method for absolute counting of CD4+ cells and CD 45 cells at a single wavelength (range).

It is yet another object of the present invention to provide a method for diagnosis of HIV infections based on the absolute counting of CD4+ cells and CD 45 cells.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a graph showing the results of analysis of the fluorescence intensities of CD4_PE and CD8_silica nanoparticles according to a preferred embodiment of the present invention;

FIG. 2 is a schematic view showing the analysis of fluorescence intensities according to the prior art;

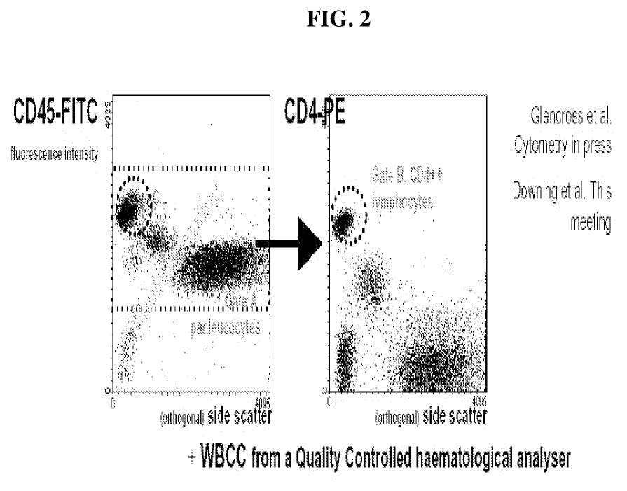

FIG. 3 is a graph showing the results of analysis of the fluorescence intensities of CD45_PE and CD4_silica nanoparticles according to a preferred embodiment of the present invention;

FIG. 4 is a graph showing the results of analysis of the fluorescence intensities of CD4_PE and CD8_silica nanoparticles according to a preferred embodiment of the present invention;

FIG. 5 is a graph showing the results of fluorescence intensities obtained by using PMT (Photo Multiplied Tube); and

FIG. 6 is a schematic view of PI (Propidium Iodide)-doped silica nanoparticles.

### DETAILED DESCRIPTION

According to preferred embodiments of the present invention, two types of fluorescent materials showing different fluorescence intensities at a single wavelength range are used to analyze and count at least two markers present in a single cell surface.

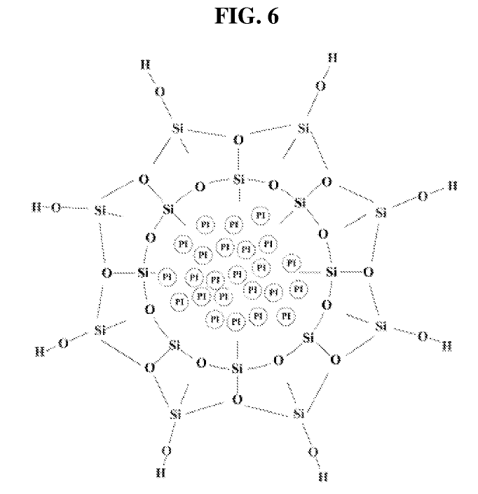

Examples of the fluorescent materials used in the embodiments, preferably, include PI (Propidium Iodide)-doped silica nanoparticles. The PI (Propidium Iodide)-doped silica nanoparticles can be prepared by a known method in the art (Adv. Mater. 2004, 16, 173-176; Anal. Chem. 2001, 73, 4988-4993; Chem. Commun. 2004, 10, 2810-2811; J. Biomed. Mater. Res.—Part A 2003, 66, 870-879) (see FIG. 6).

According to a preferred embodiment of the present invention, CD4+ T cells conjugated with PE (phycoerythrins), i.e. CD4_PE, and CD8+ T cells conjugated with PI (Propidium Iodide)-doped silica nanoparticles, i.e. CD8_silica nanoparticles are used. It is known that PE and PI show similar level of fluorescence intensity when they are excited by green light laser.

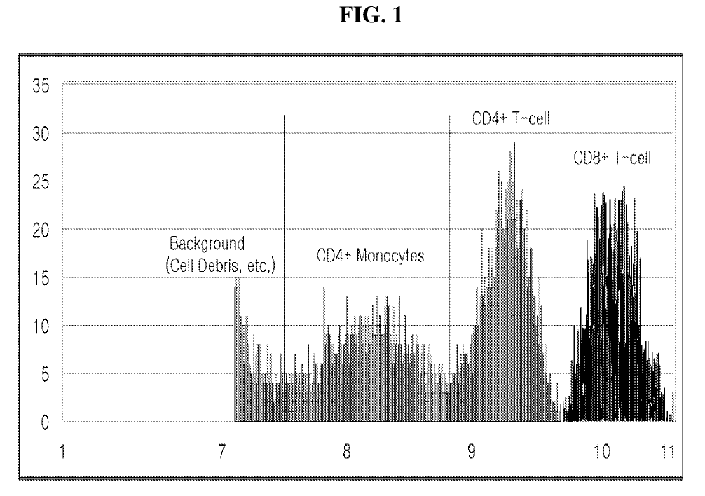

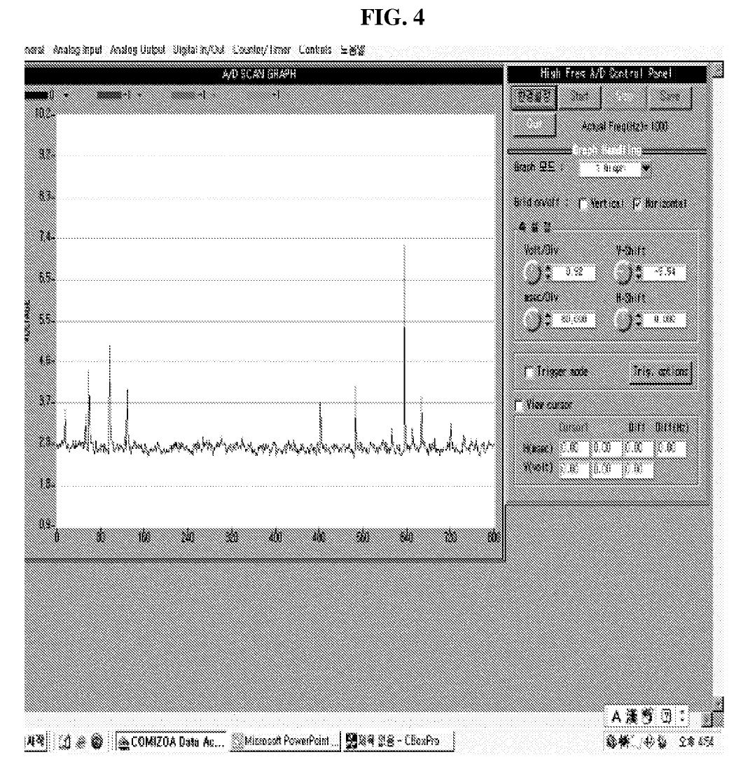

A green light is irradiated to CD4_PE and CD8_silica nanoparticles. Then wavelengths emitted therefrom are monitored. The result was that CD8+ T cells emitted light 10-100 times brighter than that emitted by CD4+ T cells. Referring to FIGS. 1 and 4, a significant difference in two histogram peaks of the intensity domain was observed. While not intending to limit a theory, it can be contemplated that this results from the use of a marker including 100-200 PI particles in silica nanoparticles.

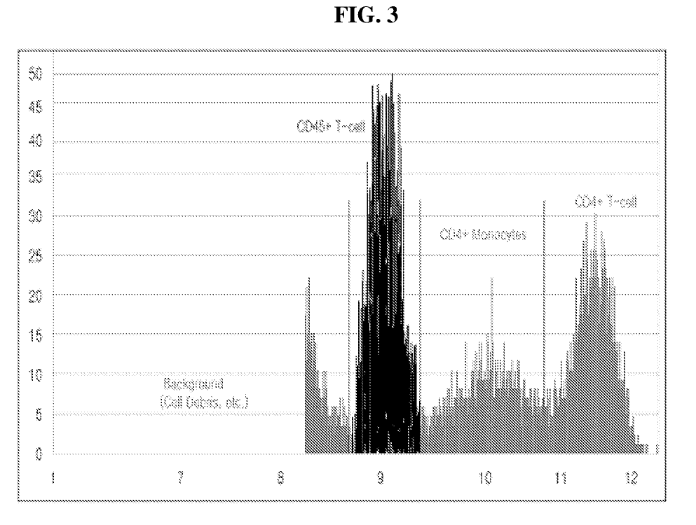

In another preferred embodiment, CD45 cells conjugated with PE (phycoerythrins), i.e. CD45_PE, and CD4 T cells conjugated with PI (Propidium Iodide)-doped silica nanoparticles, i.e. CD4_silica nanoparticles, are used to perform absolute counting of CD45 (all the white blood cells) and CD4+ T cells (see FIG. 3).

Preferred embodiments of the present invention will now be detailed by the following examples. It is to be understood that the following examples are illustrative only and the present invention is not limited thereto.

### EXAMPLE 1

### Counting of CD4+ T Cells and CD8+ T Cells Using CD4_PE and CD8_Silica Nanoparticles

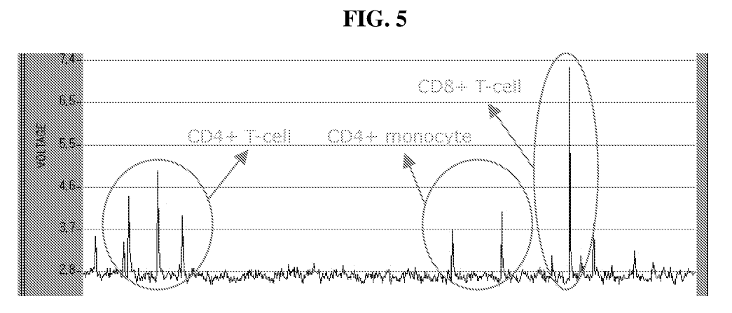

PE (phycoerythrins) was conjugated to CD4+ T cells. PI (Propidium Iodide)-doped silica nanoparticles (about 100 nm, prepared directly by the inventors of the present invention) was conjugated with CD8+ T cells. The above two samples were mixed in a ratio of 1:1, and the mixture was introduced into the whole blood to perform a reaction for 10 minutes. Next, analysis was performed by using a flow cytometer, FACS (Fluorescence Activated Cell Sorter) or a microchip based cell counter. PE and PI (Propidium Iodide)-doped silica nanoparticles emitted a red light upon the irradiation with a green laser. The red light emitted from each sample was analyzed by using PMT (Photo Multiplied Tube) to determine the intensity of the light (See FIG. 5).

The following cell counting results were obtained: 5,321 CD4+ T cells; 3,684 CD4+ monocytes; and 3,578 CD8+ T cells. In a normal human body, a ratio of about 1:0.6:0.6 is shown among the three types of cells. If the proportion of CD4+ T cells is less than 0.7, there is a possibility of HIV infection.

### EXAMPLE 2

### Counting of CD45 and CD4+ T Cells Using CD45_PE and CD4_Silica Nanoparticles

Counting of CD4+ T cells and CD 45 cells was carried out in the same manner as described in Example 1, except that CD45_PE and CD4_silica nanoparticles were used instead of CD4_PE and CD8_silica nanoparticles (see FIG. 3). On the resultant histogram, CD4+ T cells appeared in the lightest region.

The following cell counting results were obtained: 5,122 CD4+ T cells; 2,978 CD4+ monocytes; and 20,617 CD45 (all white blood cells). In a normal human body, a ratio of about 2:1:10 is shown among the three types of cells. If the proportion of CD4+ T cells is less than 0.2 (as expressed by the ratio of [CD4]/[CD45]), there is a possibility of HIV infection.

As can be seen from the foregoing, the methods according to the present invention make it possible to carry out sorting and counting of at least two markers by a flow cytometer using a single light source having a single wavelength range. Thus, the flow cytometry methods according to the present invention can be applied to a compact and inexpensive diagnostic instrument.

Although several preferred embodiments of the present invention have been described for illustrative purposes, those skilled in the art will appreciate that various modifications, additions and substitutions are possible, without departing from the scope and spirit of the invention as disclosed in the accompanying claims.

What is claimed is:

1. A method for counting a plurality of cells by flow cytometer, the method comprising the steps of:
conjugating a first fluorescent material with a first cell sought to be detected;
conjugating a second fluorescent material with a second cell sought to be detected, the second fluorescent material being doped on silica nanoparticles;
applying a light to the fluorescent materials by a light source of a single wavelength in the flow cytometer; and
detecting fluorescence intensities of light emitted from the materials.

2. The method as claimed in claim 1, wherein the first cell is CD4+ T cell and the second cell is CD8+ T cell, the CD4+ T cell being conjugated with PE (phycoertythrins), and the CD8+ T cell being conjugated with PI (Propidium Iodide)-doped silica nanoparticles.

3. The method as claimed in claim 1, wherein the first cell is CD45 cell and the second cell is CD4+ T cell, the CD45 cell being conjugated with PE (phycoertythrins), and the CD4+ T cell being conjugated with PI (Propidium Iodide)-doped silica nanoparticles.

4. A flow cytometer for diagnosis of HIV infections, which comprises a fluid containing CD4+ T cells conjugated with PE (phycoertythrins) and CD45 cells conjugated with PI (Propidium Iodide)-doped silica nanoparticles.

5. A method for counting a plurality of cell surface markers present in a single cell by flow cytometer, the method comprising the steps of:
conjugating a first fluorescent material with a first cell surface marker sought to be detected;
conjugating a second fluorescent material with a second cell surface marker sought to be detected, the second fluorescent material being doped on silica nanoparticles;
applying a light to the fluorescent materials by a light source of a single wavelength in the flow cytometer; and
detecting fluorescence intensities of light emitted from the materials.

6. The method as claimed in claim 5, wherein the first cell surface marker is CD4+ T cell and the second cell surface marker is CD8+ T cell, the CD4+ T cell being conjugated with PE (phycoertythrins), and the CD8+ T cell being conjugated with PI (Propidium Iodide)-doped silica nanoparticles.

7. The method as claimed in claim 5, wherein the first cell surface marker is CD45 cell and the second cell surface marker is CD4+ T cell, the CD45 cell being conjugated with PE (phycoertythrins), and the CD4+ T cell being conjugated with PI (Propidium Iodide)-doped silica nanoparticles.
